(19) United States
(12) Patent Application Publication
(10) Pub. No.: US 2008/0281209 A1
(43) Pub. Date: Nov. 13, 2008

(54) Optical Device

(76) Inventors: John William Arkwright, New South Wales (AU); Simon Nicholas Doe, South Australia (AU); Vinay Kumar Tyagi, Victoria (AU); Edward William Preston, New South Wales (AU)

Correspondence Address: KENYON & KENYON LLP, ONE BROADWAY, NEW YORK, NY, 10004, US

(21) Appl. No.: 11/886,131

(22) PCT Filed: Mar. 9, 2006
(86) PCT No.: PCT/AU2006/000309

§ 371 Date: May 16, 2008

(30) Foreign Application Priority Data
Mar. 10, 2005 (AU) 2005901143
Nov. 4, 2005 (AU) 2005906109

(51) Int. Cl. A61B 6/00; G02B 6/34; C03B 37/07

(52) U.S. Cl. 600/478; 385/13; 65/377

(57) ABSTRACT

The present invention provides an optical device which comprises a light guide incorporating a Bragg grating. The apparatus also comprises a moveable wall portion which is coupled to the Bragg grating so that a movement of the wall portion causes a force that effects a change in strain of the Bragg grating and thereby effects a change in an optical period of the Bragg grating. A temperature related change in the optical period of the Bragg grating is reduced by a temperature related change in the force on the Bragg grating by the moveable wall portion.

### FIELD OF THE INVENTION

The present invention broadly relates to an optical device and relates particularly, though not exclusively, to an apparatus for pressure sensing.

### BACKGROUND OF THE INVENTION

Pressure measurements are conducted in a variety of different media and for a variety of different purposes. For example, pressure is measured in open air, under water and in devices or machines. Mechanical or electronic devices typically are used for such pressure measurements.

Recently optical pressure measurement devices became popular in which an external pressure change effects a change in light interference conditions which can be detected. Such an optical device may comprise a fibre Bragg grating which has an optical response that depends on a strain of the Bragg grating. Specifically, if the strain is increased, a wavelength of a reflected light beam will shift to longer wavelengths.

Such optical devices have the advantage that they can be relatively small and may be manufactured from materials that are largely inert (such as glass) and not easily affected by many chemicals. However, temperature changes also effect a change in the interference conditions of such Bragg gratings. In general, the refractive index of such a Bragg grating will increase with increasing temperature and therefore the optical period, and hence the wavelength of the reflected beam, will also increase with increasing temperature. Consequently such optical devices can only provide reliable information about the pressure if the temperature is known. For many applications the detection of temperature changes may not be possible or convenient. There is a need for technological advancement.

### SUMMARY OF THE INVENTION

The present invention provides in a first aspect an optical device comprising:

a light guide,

a Bragg grating incorporated into the light guide,

a moveable wall portion coupled to the Bragg grating so that a movement of the moveable wall portion causes a force that effects a change in strain of the Bragg grating and thereby effects a change in an optical period of the Bragg grating,

wherein a temperature related change in the optical period of the Bragg grating is reduced by a change in the physical period of the Bragg grating caused by a temperature related change in the force by the moveable wall portion.

The optical device typically is an apparatus for pressure sensing. The moveable wall portion typically has opposite first and second sides and is positioned so that a change in pressure at one of the sides relative to a pressure at the other side will move the moveable wall portion.

The optical device typically comprises an enclosed space and the moveable wall portion typically is positioned so that a change in external pressure will move the moveable wall portion. The optical device typically comprises an enclosure having the moveable wall portion and forming the enclosed space.

In this embodiment the dual function of the moveable wall portion, namely reducing a temperature related change in the optical period of the Bragg grating and causing a force on the Bragg grating in response to an external pressure change, facilitates a compact design of the optical device.

The optical device typically has a normal operating temperature and pressure range at which the Bragg grating is distorted, typically, but not exclusively, by the force caused by the moveable wall portion. The Bragg grating typically is distorted into the enclosed space.

The light guide typically is attached to a rigid portion of the enclosure at attachment regions between which a sensing region of the Bragg grating is defined. The or each light guide typically is secured in or on the rigid portion of the enclosure so that the rigidity of the rigid portion prevents that an axial force acting on the light guide external to the enclosure affects the optical response of the Bragg grating.

The optical device typically is arranged so that the force caused by a change in external pressure is a sideway-force on the Bragg grating.

The moveable wall portion typically is a diaphragm and, at ambient temperature and pressure, typically is positioned so that the diaphragm applies the force on the Bragg grating in a manner such that the distortion of the Bragg grating into the enclosed space increases. Consequently, a temperature related change in material properties of the diaphragm, such as a property related to the Young's modulus, thermal expansion or other such properties, typically reduces the force on the Bragg grating and thereby reduces a temperature related change in strain of the Bragg grating between the attachment regions caused by a thermal expansion of the Bragg grating.

Further, a temperature increase will typically result in an increase of a pressure in the enclosed space which typically will also reduce the force applied by the diaphragm on the Bragg grating and thereby reduces a temperature related change in strain of the Bragg grating between the attachment regions.

As the temperature related change in strain of the or each Bragg grating is reduced, the pressure measurement is largely independent from changes in temperature, at least over a predetermined temperature range, which has significant practical advantages.

The optical device may be used for pressure measurements in any environment, including for example in-vivo-environments, laboratories and wind tunnels.

The optical device may comprise an external catheter that may be arranged for insertion into a human body. Further, the optical device may comprise a portion comprising an X-ray opaque material which enables imaging the position of the optical device in the human body.

The enclosure typically is arranged and the Bragg grating typically is positioned so that the optical response of the Bragg grating is a non-linear function of the temperature. In this case a plot of the optical period of the Bragg grating as a function of the temperature typically has at least one valley and may have, at least for one temperature range, a combined quadratic and linear dependency on the temperature. An optical response of the Bragg grating typically has a linear dependency on the temperature and on axial strain, but the strain on the Bragg grating attached to the enclosure typically has a quadratic dependency on the temperature. Consequently, if the Bragg grating is arranged so that a change in temperature of the enclosure also causes a change in strain, the optical response of the Bragg grating will have a combined quadratic and linear dependency on the temperature.

The normal operating temperature of the optical device may be a temperature at which the optical period has a minimum in the valley and by selecting a strain applied to the Bragg grating it is possible to select the normal operating temperature. The enclosure and the Bragg grating typically are arranged so that the optical period of the Bragg grating does not change by more than 0.001 nm if the temperature changes by ±1 degree and no more than 0.05 nm if the temperature changes by ±10 degrees from the normal operating temperature of the optical device.

The light guide with the Bragg grating may be in direct contact with the diaphragm. In one specific embodiment of the present invention the light guide with the Bragg grating is indirectly coupled to the optical device and has an anvil positioned between the diaphragm and the Bragg grating.

The Bragg grating may be positioned on the diaphragm and outside the enclosure. Alternatively, the Bragg grating may be positioned within the diaphragm or on the diaphragm and inside the enclosure.

The enclosure may comprise a casing that is formed from a rigid material and the moveable wall portion, for example provided in the form of the diaphragm, may be positioned opposite a rigid wall portion of the casing. In this case the optical device is suitable for sensing the pressure change on one side of the optical device. Alternatively, the moveable wall portion may surround a portion of the enclosed space of the enclosure. In this case the Bragg grating typically also surrounds at least a portion of the enclosed space.

In another specific embodiment the moveable wall portion and the respective Bragg grating circumferences the entire enclosed space and the optical device is arranged so that pressure changes can be sensed in a region that radially surrounds the optical device.

In one specific embodiment the optical device comprises a series of Bragg gratings with corresponding enclosures. In this embodiment, the Bragg gratings and the light guide comprise one optical fibre. The optical fibre is in this embodiment attached to the rigid portions of the respective enclosures, but is flexible at regions between two enclosures of the series so that the optical device is articulated.

The enclosure typically is filled with a compressible fluid such as air.

The light guide may comprise an optical fibre such as a single mode optical fibre in which the or each Bragg grating may have been written. As optical fibres are known to cause very little signal loss per length, the optical device can have a relatively long optical fibre lead and an optical analyser for analysing the response from the or each Bragg grating may be remote from the or each Bragg grating, such as 1 m, 10 m, 1 km or 100 km remote from the or each Bragg grating.

Alternatively, the optical device may comprise a plurality of Bragg gratings associated with a plurality of respective light guiding arms of the optical device.

The optical device may be arranged so that the optical response from the or each Bragg grating can be detected by detecting light that is reflected back from the or each Bragg grating. In this case the light guide typically is arranged so that the light is guided to and from the or each Bragg grating by the same optical fibre portion.

The optical device may also be arranged so that the optical response from the or each Bragg grating can be detected by detecting light that is transmitted through the or each Bragg grating. In this case the light guide typically comprises at least one optical fibre for guiding the light to the or each Bragg grating and at least one other optical fibre for guiding the light from the or each Bragg grating.

In one specific embodiment of the present invention the device comprises a series of Bragg gratings for distributed pressure sensing. Each Bragg grating of the series typically is arranged do give a different optical response so that light guided through the or each Bragg gratings is wavelength division multiplexed. With such a device it is possible to detect pressure changes at a series of positions which correspond to the positions of the Bragg gratings. As each Bragg grating gives a different response, it is possible to associate a particular pressure change with a respective position within the body.

In a variation of this embodiment the optical device also comprises a plurality of the Bragg gratings, but at least some of the Bragg gratings are substantially identical and typically give the same response if the strain conditions are the same. Using time division multiplexing techniques, the position of a particular Bragg grating may be estimated from a time at which an optical response is received.

In one embodiment the or each Bragg grating and the light guide comprises one optical fibre. For example, the or each Bragg grating may be written in the optical fibre and light guide may be integrally formed. Alternatively the optical fibre may comprise portions that are spliced together.

The present invention provides in a second aspect a method of fabricating an apparatus for pressure sensing, the method comprising:

providing a light guide having a Bragg grating,

selecting a design for a moveable wall portion, the moveable wall portion having opposite first and second sides,

positioning the moveable wall portion so that a change in pressure at one of the side relative to a pressure at the other side will move the moveable wall portion,

selecting a distortion for the or each Bragg grating, and

coupling the Bragg grating to the moveable wall portion so that the Bragg grating has the selected distortion and the movement of the moveable wall portion causes a force that effects a change in strain of the Bragg grating,

wherein the design of the moveable wall portion and the distortion of the Bragg grating are selected so that a temperature related change in optical period of the Bragg grating is reduced by a temperature related change in the force on the Bragg grating.

The apparatus typically is fabricated so that the apparatus has an enclosed space and the Bragg grating is distorted into the enclosed space.

The step of selecting a design of the moveable wall portion typically comprises selecting a thermal expansion coefficient of a material for forming the moveable wall portion.

The step of selecting a design of the moveable wall portion typically comprises selecting a Young's modulus for the moveable wall portion, which typically is a diaphragm.

The present invention provides in a third aspect an apparatus for pressure sensing fabricated by the above-defined method.

The invention will be more fully understood from the following description of specific embodiments of the invention. The description is provided with reference to the accompanying drawings.

### BRIEF DESCRIPTION OF THE DRAWINGS

FIGS. 1 (a) and (b) shows a sensing system according to a specific embodiment of the present invention,

FIG. 2 (a) and FIGS. 2 (a) and (b) show an optical device according to an embodiment of the present invention and FIG. 2 (c) shows an alternative component of the apparatus for pressure sensing,

FIG. 3 shows a plot of Bragg grating responses as a function of temperature,

FIGS. 4 (a) and (b) shows an optical device according to a specific embodiment of the present invention,

FIGS. 5 (a) and (b) shows an sensing apparatus according to a further specific embodiment of the present invention,

FIG. 6 shows an optical device according to another specific embodiment of the present invention and

FIG. 7 shows an optical device according to yet another specific embodiment of the present invention.

### DETAILED DESCRIPTION OF SPECIFIC EMBODIMENTS

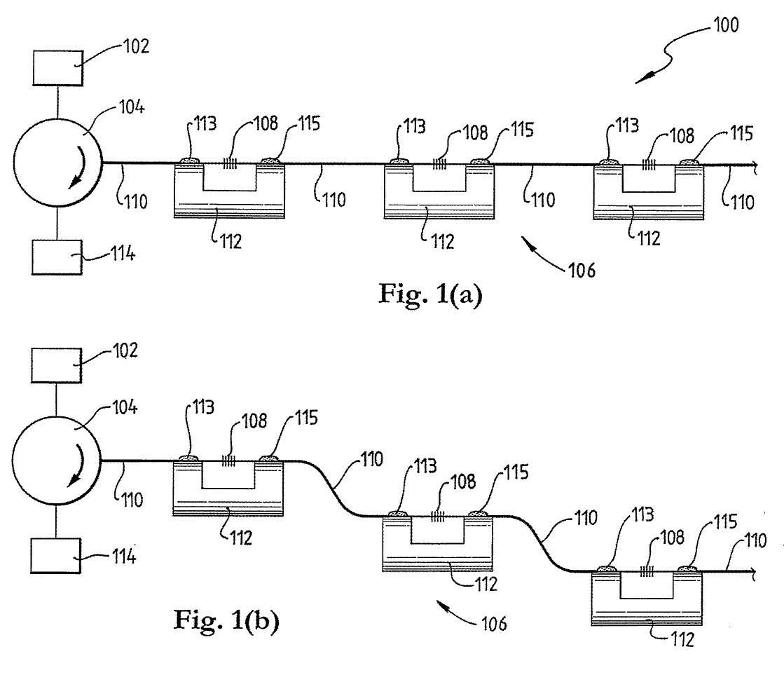

FIGS. 1, 2 and 3-7 show embodiments of the optical device or sensing system in which the optical device is an apparatus for pressure sensing. It is to be appreciated, however that the present invention has broader applications and the optical device may not necessarily be a pressure sensing apparatus.

Referring initially to FIG. 1 (a), a system for pressure sensing according to a specific embodiment of the present invention is now described. The system 100 comprises a light source 102 which in this embodiment is a broadband light source commonly referred to as a “white” light source even though the light that is emitted by the light source 102 may have any wavelength range.

The light is directed via optical circulator 104 to an apparatus for pressure sensing 106. In a variation of this embodiment the circulator 104 may be replaced by an optical coupler, an optical splitter or an optical beam splitter.

The apparatus 106 may comprise a catheter (not shown) for insertion into the human body. Further, the apparatus 106 typically comprises an X-ray opaque material, such as a metallic material, for locating the apparatus 106 in the human body.

In this embodiment the apparatus 106 comprises a series of Bragg gratings 108 which are formed in an optical fibre and which are linked by optical fibre portions 110. Each Bragg grating 108 is in this embodiment positioned in association with an enclosure 112. Each enclosure 112 has a movable wall portion which is provided in the form of a diaphragm (not shown). In this embodiment, the optical fibre 110 is rigidly connected at end-portions 113 and 115 of a respective enclosure 112 so that a respective Bragg grating 108 is positioned between two end portions. Each Bragg grating is positioned on or near a respective diaphragm such that an external pressure change effects movement of the diaphragm which in turn will apply a strain to the Bragg grating 108. The strain causes a change of an optical property of the Bragg grating 108, such as a change of an optical path length, which influences an optical response of the grating 108 to light guided to the Bragg grating 108. Consequently it is possible to sense a pressure change from analysing the optical response from the Bragg gratings.

It will be appreciated, that in alternative embodiments each Bragg grating 108 may be positioned within or below a respective diaphragm. The remaining walls of the enclosure 112 are formed from a rigid material, such as silicon, a plastics or metallic material (for example stainless steel, invar, tungsten, or kovar), or any other suitable rigid material. In this embodiment the apparatus 106 comprises a series of three Bragg gratings 108. In alternative embodiments the apparatus 106 may comprise any other number of Bragg gratings at any fixed or variable pitch.

In this embodiment each Bragg grating 108 of the series has a slightly different refractive index variation so that each Bragg grating 108 has an optical response that has a slightly different spectral response. The light that is produced-by light source 102 and that is directed to the Bragg gratings 108 therefore causes three unique responses from the Bragg gratings 108 which are directed via the optical circulator 104 to optical analyser 114 for optical analysis. Such a procedure is commonly referred to as wavelength division multiplexing (WDM). The Bragg grating may also effect optical responses which overlap in wavelength or frequency space as long as sufficient information is known about each Bragg grating to allow the signals to be successfully deconvolved.

As in this embodiment each Bragg grating 108 causes a different response, it is possible to associate a particular response with a position along the apparatus 106. Consequently it is possible to perform distributed pressure measurements and detect relative pressure difference between the positions of the Bragg gratings 108 in the series. The combined response from the Bragg gratings is wavelength division multiplexed and the optical analyser 114 uses known wavelength division de-multiplexing techniques to identify the responses from the respective grating positions. Suitable software routines are used to determine a pressure or pressure distribution from the optical responses received from the Bragg gratings. Pressure measurements typically include calibrating the apparatus.

In a variation of this embodiment at least some of the Bragg gratings 108 may be identical and consequently, if the strain conditions are the same, their optical response will also be the same. In this case a pulsed light source may be used to guide light to the Bragg gratings and the positions of the Bragg gratings may be estimated from a time at which the responses are received by the optical analyser 114.

In one particular example the reflectivity of each Bragg grating 108 is chosen so that each response has, at the location of the optical analyser 114, approximately the same intensity.

It will be appreciated that in a further variation of this embodiment the apparatus may be arranged so that responses from respective Bragg gratings can be analysed by receiving light that is transmitted through the Bragg gratings 108. For example, in this case the apparatus 106 typically is arranged so that light is guided from the light source 102 through the Bragg gratings 108 and then directly to the optical analyser 114.

In this embodiment each Bragg grating 108 is written into an optical fibre and spliced between fibre portions 110. It will be appreciated, that in alternative embodiments the Bragg gratings 108 and the fibre portions 110 may be integrally formed from one optical fibre. The same optical fibre may be used for writing respective refractive index variations for each grating so that spaced apart Bragg gratings are formed separated by fibre portions. In this embodiment the enclosures 112 comprise a rigid material while the fibre portions 110 are relatively flexible. Consequently the apparatus 106 is an articulated device. FIG. 1 (b) shows the system for pressure sensing 100 also shown in FIG. 1 (a), but the optical fibre 110 is bent between the enclosures 112 of the articulated device.

In variations of this embodiment the apparatus may comprise a plurality of Bragg gratings associated with respective optical fibres that are arranged in parallel.

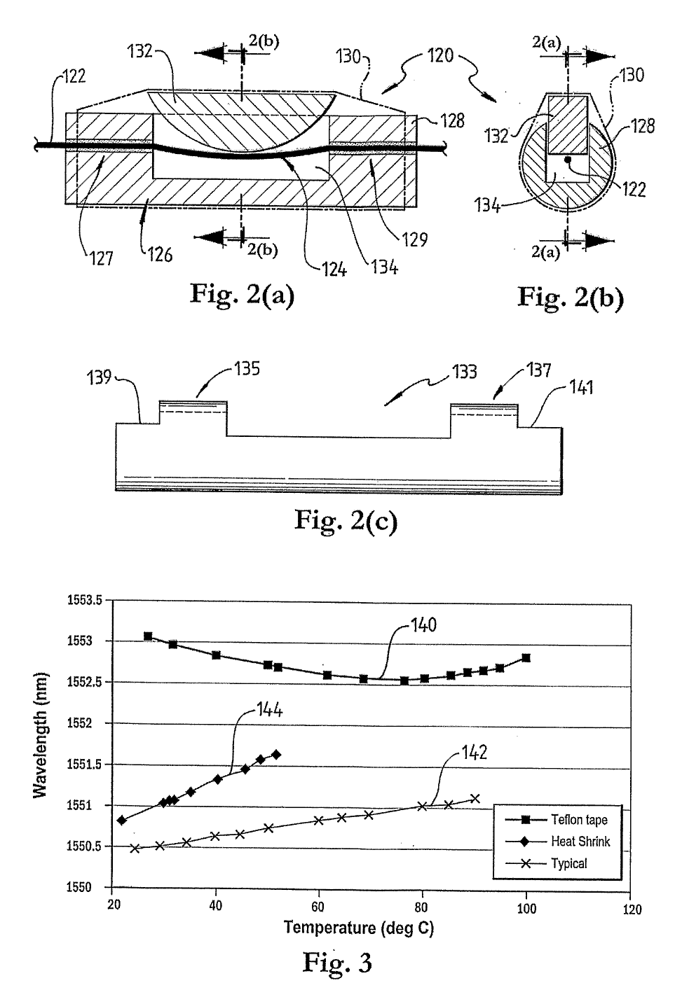

FIGS. 2 (a) and (b) show schematically an apparatus for pressure sensing in more detail. The apparatus 120 comprises an optical fibre 122, a Bragg grating 124 and an enclosure 126 which includes a body 128, a diaphragm 130 and an anvil 132. The enclosure 126 encloses a space 134 and is arranged so that a change in external pressure will. change the enclosed space 134 by deflecting the diaphragm 130 and the anvil 132 will increase the distortion of the Bragg grating 124. In this embodiment the Bragg grating 124 is distorted into the enclosed space 134 and the optical fibre 122 is attached to the enclosure 126, which is composed of a rigid material, at attachment regions 127 and 129.

In the example shown in FIGS. 2 (a) and (b) the distortion of the Bragg grating 124 causes a tensile strain of the Bragg grating 124. If the ambient temperature now increases from the normal operation temperature, a number of physical effects may take place. The optical period of the Bragg grating 124 will typically increase and the enclosed space 134 will tend to expand. Further, the diaphragm material, which typically is positioned so that the distortion of the Bragg grating is increased at a normal operating temperature, will tend to expand and/or the Young's modulus of the diaphragm material may decrease which in turn causes a decrease of the distorting force on the Bragg grating 124 and thereby counteracts the increase of the optical period. Hence, it is possible to influence the temperature dependency of optical responses by selecting materials having selected thermal behaviour.

Since typically the above physical processes influence the grating response as a function of temperature, it is possible to select a design for the apparatus a Bragg grating distortion so that the valley of the plot 140 can be shifted to wide range of temperatures. Further, it is possible to design the apparatus so that the plot 140 would have more than one valley and/or peak and hence provide an extended range over which acceptable athermal behaviour is achieved.

FIG. 2 (c) shows an enclosure 133 which is a variation of the enclosure 126 shown in FIG. 2 (a). The enclosure 133 has two portions 135 and 137 for securely fixing an optical fibre containing a Bragg grating and two recesses 139 and 141 for coupling the optical fibre in a flexible manner. The flexible coupling portions reduce bending forces at the portions 135 and 137 on the coupled Bragg grating.

It is to be appreciated that the apparatus shown in FIG. 2 has only one of many possible designs. For example, the apparatus may not necessarily have an anvil but the Bragg grating may be mechanically distorted into the enclosed space without an anvil and in contact with the diaphragm. The optical fibre 122 containing the Bragg grating 124 is in this example secured on the enclosure at positions 127 and 129 so that the Bragg grating is located between positions 127 and 129 and an optical response of the Bragg grating 124 has a partially quadratic dependency on the temperature.

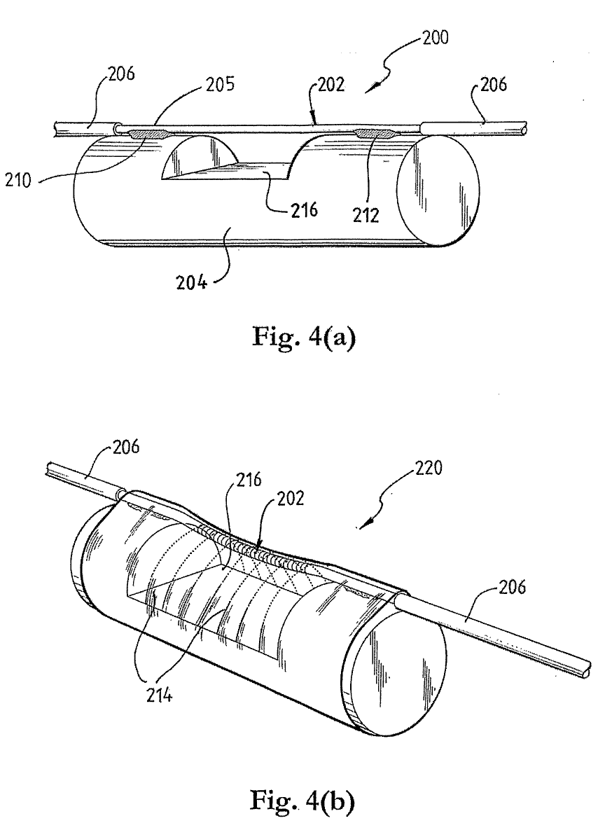

FIGS. 4 (a) and 4 (b) shows an apparatus for pressure sensing according to another embodiment of the present invention. In this embodiment the apparatus 200 comprises a Bragg grating 202 and a body 204. The Bragg grating 202 is formed in an optical fibre that comprises a core/cladding region 205 and a protective coating 206. The protective coating 206 has been stripped away in the area of the Bragg grating 202. The core/cladding region is attached to the body 204. In this embodiment the core/cladding region 205 is glued to the body 204 at regions 210 and 212. For example, the body may be formed from silicon, a plastics or metallic material, or any other suitable rigid material.

FIG. 4 (b) shows an apparatus 220, a variation of the apparatus 200, with a diaphragm 214 applied to it. For example, the diaphragm 214 may be a cold or hot shrink tube which is inserted over the Bragg grating 202 and over the body 204 or an elastic material that stretches around the body 204. As the body 204 has a recess 216, an enclosed pressure sensitive space is formed at the recess 216 and below the diaphragm 214. The diaphragm 214 is composed of a flexible material such as a rubber or nylon material, a flexible metal foil or silicone foil. Similar to the embodiment shown in FIG. 2, the Bragg grating 202 is slightly distorted into the enclosed space in the recess 216 (the distortion is indicated in FIG. 4 (b) and not shown in FIG. 4(a)).

FIG. 3 shows plots of Bragg grating responses as a function of temperature. Plot 140 shows the response of a grating of the apparatus for pressure sensing shown in FIGS. 4 (a) and (b). In this example, the enclosure 204 is formed from stainless steel and the diaphragm is formed from polyolefin heat shrink. FIG. 3 shows also a plot 142 for a typical Bragg grating that is not coupled to an enclosure and to a diaphragm and a plot 144 for a Bragg grating with the optical fibre being bonded to a stainless steel substrate and enclosed by Teflon tape (3M#60 PTFE tape).

In the example shown in FIG. 4 the optical fibre containing the Bragg grating 202 is secured on the enclosure 204 at positions adjacent the Bragg grating 202 so that the Bragg grating is located between attachment regions. An optical response of the Bragg grating 202 has a partially quadratic dependency on the temperature. The refractive index of the Bragg grating 202 is approximately linearly dependent on the strain applied to the Bragg grating 202 and the optical response of Bragg grating 202 is dependent on both the refractive index and the optical period. The normal operating temperature of the apparatus is a temperature at which the optical period has a minimum in the valley and by selecting a strain and a distortion applied to the Bragg grating 202 it is possible to select the normal operating temperature. In this example the distortion of the Bragg grating 202 and the design of the enclosure 204 are selected so that the optical response of the or each Bragg grating does not change by more than approximately 0.001 nm if the temperature changes by ±1 degree from the normal operating temperature of the apparatus which typically is of the order of 77° C.

In this example the valley is positioned at approximately 77° C., but a person skilled in the art will appreciate that in a variation of this embodiment the apparatus may be designed so that the valley is positioned at approximately 37° C., or normal body temperature, which would then be the normal operating temperature.

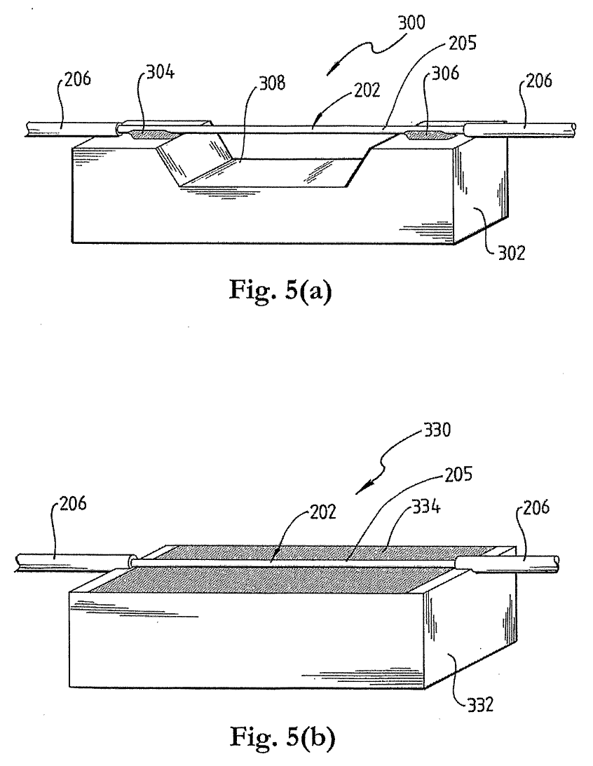

FIGS. 5 (a) and 5 (b) shows apparatus 300 and 330 according to further embodiments of the present invention. Both the apparatus 300 and the apparatus 330 comprise the Bragg grating 202, the fibre core/cladding 205 and the protective coatings 206. The apparatus 300 comprises a body 302 to which the core/cladding region 205 is glued at regions 304 and 306. In this embodiment the body 302 has a substantially rectangular cross sectional area and may be formed from silicon or any other suitable rigid material.

The device 300 further comprises a flexible cover, such as a diaphragm, (not shown) which is positioned over the Bragg grating. 202 and encloses recess 308 of the rigid structure 302. Alternatively, the cover may be positioned below the Bragg grating 202 and may cover the recess 308 so that an enclosed internal space is formed below the Bragg grating 202. In this case the Bragg grating 202 typically is coupled to the cover so that a movement of the cover causes a strain to the Bragg grating 202 and consequently a pressure change can be sensed.

The apparatus 330 shown in FIG. 5 (b) comprises a rigid casing 332 which has a flexible cover 334. The casing 332 is hollow and the flexible cover 334 closes the casing 332 to form a hollow internal space below the Bragg grating 202. As in the previous example, the flexible cover may be a diaphragm. The optical fibre containing the Bragg grating 302 is attached to the flexible cover so that a movement of the flexible cover will cause a strain in the Bragg grating. The casing 332 typically is composed of a silicon material or of any other suitable rigid material. The flexible cover 334 typically is a thin layer that provides sufficient flexibility and is composed of silicone, another polymeric material or a suitable metallic material. In one specific embodiment the structure is formed from micro-machined silicon.

The examples of the apparatus for pressure sensing shown in FIGS. 2, 4 and 5 are suitable for asymmetric pressure sensing. For example, a pressure increase located only at the rigid portions of the casings 304, 303 or 332 will typically not cause a strain to the Bragg gratings 202. FIG. 6 shows an apparatus for pressure sensing according to a further embodiment of the present invention which can be used for more symmetric pressure measurements.

The apparatus 400 comprises a rigid structure 402 having rigid upper and lower portions 404 and 406 and a rigid support portion 408 connecting the upper and lower portions 404 and 406. The rigid support portion is surrounded by a diaphragm 410 which is applied to the upper and lower portions 404 and 406 so that an enclosed internal space is formed. The apparatus 400 also comprises a Bragg grating 412 and a core/cladding region 414. The core/cladding region 414 is attached to the upper and lower portions 404 and 406 at positions 418 and 420. In this embodiment the core/cladding region is glued at these positions to the upper and lower portions 404 and 406 respectively, and attached to the diaphragm 410.

For example, the optical fibre with the Bragg grating 412 may be attached to the diaphragm 410 using a flexible adhesive. If a pressure in a region adjacent the diaphragm 410 changes, the diaphragm 410 will move which will cause a strain in the Bragg grating 412 and therefore the pressure change can be sensed. As the optical fibre with Bragg grating 412 is wound around the diaphragm 410 and the diaphragm 410 surrounds the support 408 so that internal space is formed between the support 408 and the diaphragm 410, a pressure change can be sensed at any position around the diaphragm 410 using the device 400. Similar to the embodiments discussed before, the Bragg grating 412 is slightly distorted into the enclosed space (the distortion is not shown in FIG. 6).

The rigid portion 402, 404 and the support 408 typically is composed of silicon or of any other suitable rigid material including plastics or metallic materials. The diaphragm 410 typically is a thin layer having a thickness of the order of 0.1 mm being composed of silicone, another polymeric material or a metallic material.

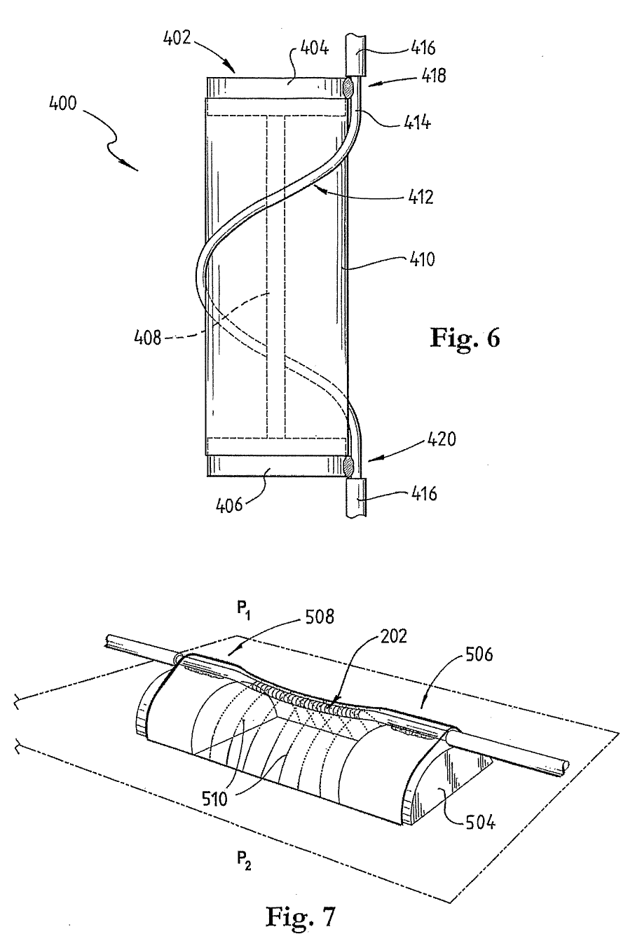

The hereinbefore-described apparatus for pressure sensing according to different embodiments of the present invention comprises an enclosure that defines an enclosed space and of which the diaphragm forms a part. In a variation of these embodiments, the apparatus for pressure sensing may not comprise such an enclosure and FIG. 7 shows an example of such an alternative design. FIG. 7 shows an apparatus for pressure 500 having an optical fibre with the Bragg grating 202 and which is attached to rigid member 504 at attachment regions 506 and 508. Diaphragm 510 distorts the Bragg grating at a normal operating temperature and separates a first region having a first pressure P₁ from a second region having a second pressure P₂. A relative change in the pressures P₁ and P₂ will move the diaphragm 510 and thereby cause a change in a force on the Bragg grating 202. As in the above-described embodiments, the diaphragm 510 and the Bragg grating 202 are positioned so that a temperature related change in optical response of the Bragg grating 202 is reduced by a temperature related change in the force on the Bragg grating. For example, the apparatus for pressure sensing 500 may be positioned across a conduit, such as a tube, for measuring a pressure caused by a flow of a fluid.

Although the invention has been described with reference to particular examples, it will be appreciated by those skilled in the art that the invention may be embodied in many other forms. For example, the apparatus for pressure sensing may comprise Bragg gratings that are positioned within the diaphragms. Further, the rigid bodies may have any suitable shape with which an enclosed internal space can be formed when a diaphragm is applied to it.

It is to be appreciated that the optical device may not necessarily be an apparatus for pressure sensing. The optical device may not comprise an enclosure that encloses a space and the moveable wall portion may not be arranged to move in response to an external pressure change. The optical device may, for example, have open ends which allow air, or any other fluid, to circulate along each side-portion of the moveable wall portion. In this instance, the temperature response of the optical device will typically be due to one or more of the thermal properties of the body, fibre and diaphragm and will not depend on any expansion of an enclosed space.

In general, the optical device may be any type of filtering, sensing or gauging device comprising a Bragg grating and wherein the moveable wall portion is arranged to reduce a temperature related change in an optical response of the Bragg grating by a temperature related change in a force on the Bragg grating. Specific examples for the optical device include spectral filters, spectral band pass filters spectral band reject (or reflection) filters, band selection filters, spectral gain filters, spectral profile filters pulse compression filters, channel dropping filters, channel blocking filters and also strain gauges.

1-33. (canceled)

34. An optical device comprising:
a light guide,
a Bragg grating incorporated into the light guide,
a moveable wall portion coupled to the Bragg grating so that a movement of the moveable wall portion causes a force that effects a change in strain of the Bragg grating and thereby effects a change in an optical period of the Bragg grating,
wherein a temperature related change in the optical period of the Bragg grating is reduced by a change in the physical period of the Bragg grating caused by a temperature related change in the force by the moveable wall portion.

35. The optical device of claim 34 being an apparatus for pressure sensing wherein the moveable wall portion has opposite first and second sides and is positioned so that a change in pressure at one of the sides relative to a pressure at the other side will move the moveable wall portion.

36. The optical device of claim 35 comprising an enclosure defining an enclosed space, the moveable wall portion forming a part of the enclosure and being positioned so that a change in external pressure will move the moveable wall portion.

37. The optical device of claim 36 having a normal operating temperature and pressure range at which the Bragg grating is distorted by the force caused by the moveable wall portion.

38. The optical device of claim 36 having a normal operating temperature and pressure range at which the Bragg grating is distorted into the enclosed space by the force caused by the moveable wall portion.

39. The optical device of claim 36 wherein the light guide with the Bragg grating is attached to a rigid portion of the enclosure at attachment regions between which a sensing region of the Bragg grating is defined.

40. The optical device of claim 39 wherein the light guide is secured in or on the rigid portion of the enclosure so that the rigidity of the rigid portion prevents that an axial force acting on the light guide external to the enclosure affects the optical response of the Bragg grating.

41. The optical device of claim 36 wherein the moveable wall portion is a diaphragm and the enclosure is arranged and the Bragg grating is positioned so that the optical response of the Bragg grating is a non-linear function of the temperature.

42. The optical device of claim 36 wherein the enclosure and the Bragg grating are arranged so that the optical period of the Bragg grating does not change by more than 0.001 nm if the temperature changes by +/−1 degree and no more than 0.05 nm if the temperature changes by +/−10 degrees from a normal operating temperature of the apparatus.

43. The optical device of claim 34 wherein the light guide with the Bragg grating is in direct contact with the moveable wall portion.

44. The optical device of claim 36 wherein the light guide with the Bragg grating is indirectly coupled to the moveable wall portion and the Bragg grating.

45. The optical device of claim 36 wherein the Bragg grating is positioned on the diaphragm and outside the enclosure.

46. The optical device of claim 36 wherein the Bragg grating is positioned within the diaphragm.

47. The optical device of claim 36 wherein the Bragg grating is positioned on the diaphragm and inside the enclosure.

48. The optical device of claim 36 wherein the enclosure comprises a casing that is formed from a rigid material and the movable wall portion is positioned opposite a rigid wall portion of the casing.

49. The optical device of claim 36 wherein the moveable wall portion surrounds a portion of the enclosed space of the enclosure.

50. The optical device of claim 36 wherein the moveable wall portion and the Bragg grating circumferences the entire enclosed space.

51. The optical device of claim 34 comprising a series of Bragg gratings with corresponding enclosures and being arranged for distributed pressure sensing.

52. The optical device of claim 34 being arranged so that the force caused by a change in external pressure is sideway-force on the or each Bragg grating.

53. The optical device of claim 34 comprising an external catheter.

54. The optical device of claim 34 comprising a portion comprising an X-ray opaque material.

55. A method of fabricating an apparatus for pressure sensing, the method comprising:
providing a light guide having a Bragg grating,
selecting a design for a moveable wall portion, the moveable wall portion having opposite first and second sides,
positioning the moveable wall portion so that a change in pressure at one of the side relative to a pressure at the other side will move the moveable wall portion,
selecting a distortion for the or each Bragg grating, and
coupling the Bragg grating to the moveable wall portion so that the Bragg grating has the selected distortion and the movement of the moveable wall portion causes a force that effects a change in strain of the Bragg grating,
wherein the design of the moveable wall portion and the distortion of the Bragg grating are selected so that a temperature related change in optical period of the Bragg grating is reduced by a change in the physical period of the Bragg grating caused by a by a temperature related change in the force by the moveable wall portion.

56. The method of claims 55 wherein the apparatus is fabricated so that the apparatus has an enclosed space and the Bragg grating is distorted into the enclosed space.

57. The method of claim 55 wherein the step of selecting a design of the moveable wall portion comprises selecting a thermal expansion coefficient of a material for forming the wall portion.

58. The method of claim 55 wherein the step of selecting a design of the wall portion comprises selecting a Young's modulus for the moveable wall portion.

59. The method of of claim 55 wherein the step of selecting a design of the moveable wall portion comprises selecting a thermal expansion coefficient for the moveable wall portion.

60. An apparatus for pressure sensing fabricated by the method of claim 55.

61. A method of measuring a pressure in an in-vivo environment, the method comprising:
inserting an apparatus for pressure sensing into a body, the apparatus comprising a light guide and a Bragg grating incorporated into the light guide,
exposing the apparatus to a pressure in the in-vivo environment so that the pressure causes a force on the Bragg grating which changes a strain of the Bragg grating and thereby changes an optical period of the Bragg grating,
reducing a temperature related change in the optical period of the Bragg grating by a change in the physical period of the Bragg grating caused by a temperature related change in the force on the Bragg grating,
guiding light to and from the Bragg grating and
receiving a response from the Bragg grating.

62. The method of claim 61 comprising the step of converting optical data into pressure data.

63. A method of measuring a muscular pressure in an in-vivo environment comprising the method as claimed in claim 61.

64. A method of measuring a muscular pressure in the oesophagus comprising the method as claimed in claim 61.

65. A method of measuring a pressure in an in-vivo environment using the optical device as claimed in claim 34.

66. An optical device comprising:
a light guide,
a Bragg grating incorporated into the light guide,
a moveable wall portion coupled to the Bragg grating so that a movement of the moveable wall portion causes a force that effects a change in strain of the Bragg grating and thereby effects a change in a optical period of the Bragg grating;
an enclosure defining an enclosed space, the moveable wall portion forming a part of the enclosure and being positioned so that a change in external pressure will move the moveable wall portion,
wherein the apparatus has a normal operating temperature and pressure range at which the Bragg grating is distorted into the enclosed space by the force caused by the moveable wall portion and wherein a temperature related change in the optical period of the Bragg grating is reduced by a temperature related change in the force on the Bragg grating by the moveable wall portion.
